(19) United States
(12) Patent Application Publication
(10) Pub. No.: US 2025/0169963 A1
(43) Pub. Date: May 29, 2025

(54) EXPANDABLE AND CONFORMING SPINAL IMPLANT

(71) Applicant: Nexus Spine, LLC, Salt Lake City, UT (US)

(72) Inventors: David T. Hawkes, Salt Lake City, UT (US); Peter A. Halverson, Salt Lake City, UT (US); Justin G. Heap, Salt Lake City, UT (US)

(21) Appl. No.: 18/963,046

(22) Filed: Nov. 27, 2024

Related U.S. Application Data
(60) Provisional application No. 63/603,558, filed on Nov. 28, 2023.
(60) Provisional application No. 63/550,491, filed on Feb. 6, 2024.
(60) Provisional application No. 63/674,445, filed on Jul. 23, 2024.

(51) Int. Cl. A61F 2/44

(52) U.S. Cl. CPC A61F 2/442; A61F 2210/00; A61F 2250/0007

(57) ABSTRACT

Systems and methods of providing an expandable and conforming interbody implant are described. According to some implementations, an interbody implant includes a first surface configured to be disposed adjacent to a first bony endplate, and a second surface configured to be disposed adjacent to a second body endplate, with a height of the interbody implant extending between the first surface and the second surface. According to some implementations, the interbody implant is configured to be selectively disposed in an insertion configuration and a deployed configuration. In some cases, the height of the interbody implant is greater in the deployed configuration than in the insertion configuration. Moreover, in some cases, the first surface is configured to conform to a topology of the first bony endplate, and the second surface is configured to conform to a topology of the second bony endplate.

### RELATED APPLICATIONS

This application claims priority to U.S. Provisional Patent Application No. 63/603,558, entitled EXPANDABLE AND CONFORMING SPINAL IMPLANT, filed Nov. 28, 2023 (Attorney Docket No. 23845.163). This application also claims priority to U.S. Provisional Patent Application No. 63/674,445, entitled IMPLANT WITH CONFORMING REGION, filed Jul. 23, 2024 (Attorney Docket No. 23845.181). This application further claims priority to U.S. Provisional Patent Application No. 63/550,491, entitled SYSTEMS AND METHODS FOR PROVIDING AND DEPLOYING INTERBODY IMPLANTS, filed Feb. 6, 2024 (Attorney Docket No. 23845.177). The contents of each of the aforementioned applications are incorporated herein by reference in their entireties.

### FIELD

The present disclosure relates to medical devices, and more particularly to spinal or other orthopedic implants.

### BACKGROUND

Traditionally, it is difficult to properly place a spinal interbody implant for a variety of reasons. For example, the interbody space to be filled is typically larger than the surgical access window allowed by surrounding anatomical structures.

Additionally, a goal of implanting an interbody device is oftentimes to increase a height of an interverbal space. However, it can be difficult to assess which interbody height would best fill the interbody space. Therefore, the selected interbody implant may be too tall, thereby applying excessive, painful, and traumatic force to spinal vertebrae and surrounding tissues, or the interbody implant may be too short, thereby presenting a risk of expulsion, migration, and painful instability.

Additionally, some traditional implants do not conform well to natural structures, such as bony regions. In this regard, they are typically too rigid to take on the shape of the mating anatomy. This can result in point loading in some regions and voids in other regions. For example, some traditional spinal implants do not always conform well to a natural bony endplate of a spinal segment of a patient. Such conformity failures can result in voids or gaps that are slow to backfill, localized regions of high contact stress that lead to bone loss and subsidence, and other serious issues that can cause instability, loss of function, pain, and decreased quality of life for patients.

Some implants create regions of hyperloading, or excessive strain, which can result in micro or macro fractures. Similarly, some implants create regions of hypoloading, which can lead to bone atrophy and prevent bone healing. In either case, these shortcomings can lead to a patient experiencing large amounts of pain, reduced physical capacity, and other major problems.

Thus, while techniques currently exist that are used to provide spinal implants, challenges still exist, including those listed above. Accordingly, it would be an improvement in the art to augment or even replace current techniques with other techniques.

### SUMMARY

When a spinal implant is applied, implant-to-bone contact can be critical for the healthy formation of bone. Such contact can impact many factors, such as stability, contact stress, rate of bone formation, mechanical stimulus of bone for continued health, expulsion, and other factors. In prior systems, implants are often selected that are too tall. This can cause numerous problems, such as subsidence due to excessive contact force, endplate violation during impaction, and other problems. Accordingly, systems and methods for providing an expandable and conforming spinal implant are disclosed herein. In some implementations, the described expandable and conforming spinal implant avoids the problems present in some prior systems.

As mentioned above, some implementations of the implant are configured to be expandable. In this regard, some iterations of the implant have an insertion configuration, in which the implant has a lower profile and is configured to be inserted into an interbody space, and a deployed configuration, in which at least a portion of the height of the implant is increased to provide the necessary support for the corresponding bony endplates of a patient's spine. Moreover, some implementations of the implant are configured to be conforming, allowing a caudal or cranial surface to conform to one or more of the specific contours and topology of an adjacent bony endplate.

Some implementations of the implant are configured to be conforming with a contact force limit. In this regard, the contact force limit can be provided such that the incidence of high-pressure contact points of the implant on the endplate, as well as substantial gaps between the implant and the endplate, are reduced to levels below the fracture limit of a vertebral endplate, or trabecular bone, or other relevant anatomical structures, such as ligaments, facets capsules, etc. Thus, in some implementations, a more even and consistent support structure for the endplate is provided.

In some implementations, the implant has a modular structure that includes multiple columns, which, in some cases, include one or more fixed columns and one or more expanding columns. Together, fixed columns and expanding columns can provide the implant with the ability to expand to the deployed configuration, conform with endplate topology, and still provide stability and structural support.

Where the implant includes expanding columns, some implementations of the expanding columns are equipped with adjustable components, allowing them to increase in height to expand the implant and apply a controlled load to the bony endplates. In some cases, these expanding columns include one or more regions with varying characteristics, such as stiffness, porosity, and material properties-enabling them to provide tailored support and adjustability. For instance, some instances of outer regions include higher stiffness for structural support, while some instances of inner regions include one or more expandable components to facilitate deployment (e.g., expansion) of the implant.

Where the implant includes fixed columns, some implementations of the fixed columns provide consistent structural support. In some cases, they are used alongside expanding columns to stabilize the implant after deployment. For example, expanding columns are (in some cases) coupled to fixed columns to transfer the load of the endplates through the fixed columns.

Although some embodiments of the fixed columns are rigid, some other embodiments of the fixed columns are not; rather, they are fixed in a relative sense when compared to the expanding columns. Still, some embodiments of the fixed columns are compressible, with a stiffness at or below the stiffness of the surrounding bone to allow for bone loading. Also, some embodiments of the fixed columns are porous.

While the expanding columns can have any suitable characteristic, in some embodiments, they are configured to limit the contact pressure on the surrounding bone to less than 1 GPa, or even less than 0.1 GPa. Although limiting contact pressure can serve any suitable purpose, in some cases, the intent of limiting the contact pressure is to prevent bone fracture or bone damage.

Many other iterations and implementations are possible, and the features and benefits of the instant systems and methods are explained and illustrated in much greater detail in the following disclosure and in the corresponding figures.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1A shows an X-ray image of a patient's spine with an interbody implant disposed therein, in accordance with an illustrative example of patient anatomy;

FIG. 1B shows an X-ray image of a patient's spine with a collapsed disc space, in accordance with another illustrative example of patient anatomy;

FIG. 1C shows an image of a bony endplate of a spinal segment of a patient to illustrate a topology thereof, in accordance with another illustrative example of patient anatomy;

FIG. 1D shows an X-ray image of a patient's spine to illustrate how rigid body implants can create gaps between implant endplates and bony endplates of the patient's spinal segments, in accordance with another illustrative example of patient anatomy;

FIG. 2A shows a side perspective view of an expandable and conforming interbody spacer, in accordance with a representative embodiment of the present systems and methods;

FIG. 2B shows a top perspective view of the expandable and conforming interbody spacer, in accordance with some representative embodiments;

FIGS. 3A and 3B each show a front elevation view of the expandable and conforming interbody spacer implant, in each case abutting a bony endplate of a spinal segment of a patient, in accordance with some representative embodiments;

FIGS. 3C and 3D each show a cross-sectional elevation view of the expandable and conforming interbody spacer implant, in each case abutting a bony endplate of a spinal segment of a patient, in accordance with some representative embodiments;

FIG. 3E shows a rear elevation view of the expandable and conforming interbody spacer implant abutting a bony endplate of a spinal segment of a patient, in accordance with some representative embodiments;

FIG. 4A shows some expanding columns as may be used in the expandable and conforming interbody spacer implant, in accordance with some representative embodiments;

FIGS. 4B and 4C each show some columns, including expanding columns and fixed columns, as may be used in the expandable and conforming interbody spacer implant, in accordance with some representative embodiments;

FIGS. 5A-5E show diagrams illustrating various configurations of columns, as may be used in the expandable and conforming interbody spacer implant, in accordance with some representative embodiments;

FIG. 6A shows a side elevation view of an example coupling mechanism for selectively coupling one column to another column, in accordance with some representative embodiments;

FIG. 6B shows a plan view of an example coupling mechanism for selectively coupling one column to another column, in accordance with some representative embodiments;

FIGS. 7A and 7B show plan views of examples of column arrangements for columns of interbody spacer implants, in accordance with some representative embodiments; and

FIGS. 8A and 8B each show a front perspective view of the expandable and conforming interbody spacer implants, according to some representative embodiments.

### DETAILED DESCRIPTION

A description of embodiments will now be given with reference to the figures. It is expected that the present systems and methods may take many other forms and shapes. Hence, the following disclosure is intended to be illustrative and not limiting, and the scope of the disclosure should be determined by reference to the appended claims.

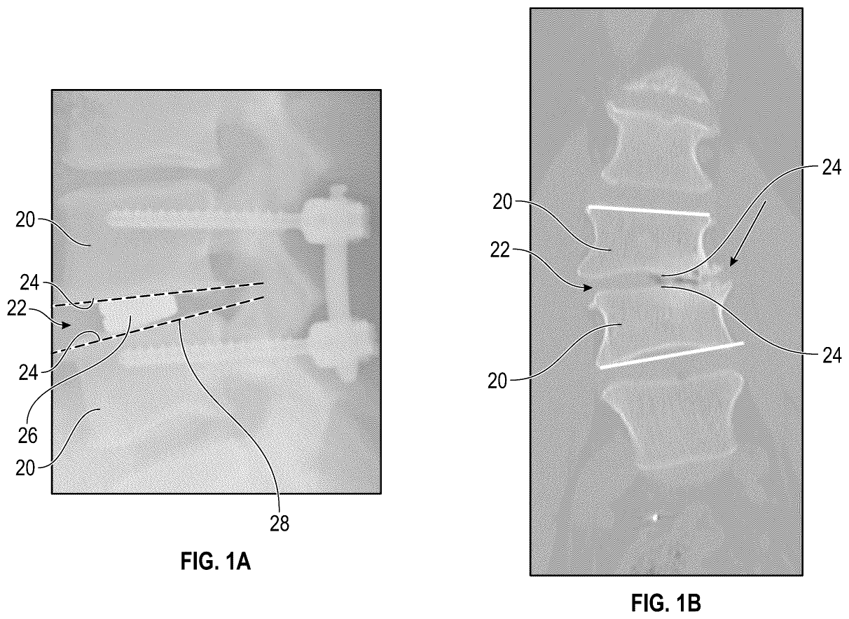

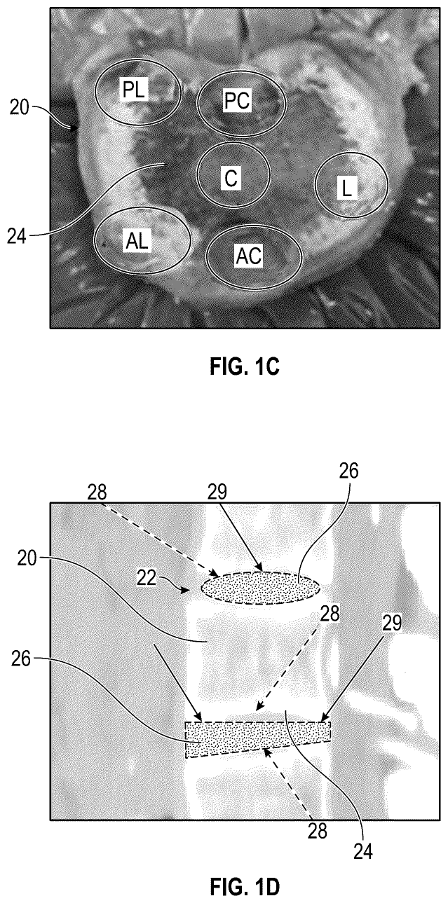

Interbody implants (also known as interbody spacers) are generally configured to be inserted between two vertebral bodies 20 of a patient's spine, into an intervertebral space 22. The implants are typically further configured to contact the bony endplates 24 of the adjacent vertebral bodies. For example, FIG. 1A shows a rigid implant 26 disposed in an intervertebral space 22 between the bony endplates 24 of adjacent spinal vertebrae 20.

Unfortunately, in some cases, an intervertebral space 22 can collapse (e.g., the endplates can shift toward each other and even, in some cases, contact each other). For example, FIG. 1B shows a pair of vertebral bodies 20 that have shifted toward each other, their endplates 24 contacting each other, thereby drastically shrinking the intervertebral space. When this happens, it can be extremely difficult to insert an implant, particularly an implant that is tall enough to provide the proper spacing between the vertebral bodies.

Moreover, some traditional spinal interbody implants (e.g., a rigid implant 26) do not always conform well to a patient's natural endplates 24 of the patient's vertebrae 20. Such conformity failures can result in voids or gaps that are slow to backfill, localized regions of high contact stress that lead to bone loss and subsidence, and other serious issues that can cause loss of function, pain, and decreased quality of life for patients. To further explain the foregoing, bony endplates of vertebral bodies typically each have their own unique topology. Such topology can vary greatly between endplates across patients and even across spinal segments of a single patient. For example, the topology of a bony endplate often forms a bowl shape, a plate shape, a parabolic dish shape, or another curved configuration. Such curvature, however, can vary substantially from one endplate to another and even within a single endplate. In this regard, the topology of a given endplate generally has a depth (or a distance from a most raised portion to a most depressed portion of the endplate), which may often range from anywhere between 0.01 mm and 4 mm (or even up to 10 mm, or any subrange between 0.01 mm and 10 mm). By way of example, a cranial (upper) endplate may have a depth of about 3.6 mm, while the topology of a caudal (lower) endplate may have a depth of about 2.6 mm. Meanwhile, another vertebra (e.g., a vertebra of another patient or another vertebra within the same patient) may have a caudal endplate with a depth of about 0.4 mm, and a rostral endplate with a depth of about 4.1 mm. Many other depths and combinations are also possible.

In some cases, in addition to having a particular depth, the topology of an endplate 24 fits into a particular topological category, such as shallow-shallow, shallow-flat, deep-shallow, deep-flat, or one or more other categories. In some cases, the topology does not necessarily fit well into a particular category. Moreover, the topology can also have regional variations. Generally speaking, a position on a topology of an endplate can be identified with the terms central (toward a midline of the endplate), lateral (away from the midline), posterior (toward a back of the patient), and anterior (toward a front of the patient). Thus, specific regions can be identified, as shown in FIG. 1C, such as central (C), lateral (L), posterior-central or (PC), anterior-central (AC), posterior-lateral (PL), or anterior-lateral (AL). Often, each region has its own natural variations (e.g., different depths, surface features, shapes, or other variations), thereby leading to a highly diverse possible range of topologies. Generally, the vertebral endplates are lenticular and the cranial side of the intervertebral space has more concavity than does the caudal side. Moreover, the amount of concavity can vary from one vertebral level to the next.

In some cases, a surgeon may need to revise a previous surgery, remove a previous implant, and repair damage to the bone and surrounding anatomy. For example, a previous implant could have subsided into the vertebral bodies and created a void in the bone that would need to be filled. In some such cases, the described conforming implant can be configured to fill the bone damage and repair the iatrogenic defect. Moreover, in such a case, additional volume and displacement of the expanding and fixed structures to conform to the void can, in some embodiments, be advantageous.

In short, each endplate 24 tends to have its own unique topology, with its own particular microscopic and macroscopic variations. Accordingly, many traditional spinal implants—which generally have no ability (or only limited ability) to conform to topological variations—often do not fit well with different endplates. Indeed, as shown in FIG. 1D, rigid or non-conforming implants 26 tend (in some cases) to create regions of hyperloading, or excessive strain, such as a contact point 29 where a portion of an endplate 20 directly contacts the rigid implant. Hyperloading can result in micro or macro fractures in the patient's spinal segments, causing pain and potentially leading to complications requiring future surgery. Similarly, some existing implants also tend to create regions of hypoloading, such as where there are gaps 28 between the implant and the body endplate (e.g., due to the endplate's depth 24). Hypoloading can lead to bone atrophy, instability of the implant, or other issues. In short, many traditional implants do not create even loading across spinal endplates having varying topologies. These shortcomings can lead to a patient experiencing large amounts of pain, reduced physical capacity, and other major problems.

Furthermore, healthy bone strength is generally no more than 8-14 N/mm², and osteopenic bone strength is typically even less than that. Indeed, in some cases, healthy vertebral bone stiffness is typically between 100 MPa and 600 Mpa (N/mm²). As a result, an implant of appropriate stiffness could potentially exceed the failure load of the endplate after ˜0.1 mm of expansion. Accordingly, it would, in some embodiments, be desirable to have an implant that can expand at a different, lower stiffness than would be ideal for its routine, daily loading.

Furthermore, by having the implant conform to the shape, or near the shape, of the bone, the implant is, in some embodiments, more capable of being retained within the interbody space by the natural endplate (e.g., as a convex shape in a concave shape is better retained than a flat shape on a concave shape).

To address these and other problems, some embodiments of the systems and methods described herein include one or more expandable and conforming interbody spacer implants 30 (sometimes referred to herein simply as implants or the implant).

According to some embodiments, the implant 30 is configured to expand (e.g., to increase a height of the implant extending between a first bony endplate 24 of a first vertebral body 20 and a second bony endplate of a second vertebral body). Accordingly, some embodiments of the implant are configured to be inserted into an intervertebral space 22 while the implant is in an insertion configuration. In some cases, the implant is condensed, compressed, or low-profile, or otherwise has a smaller height when it is in the insertion configuration such that it can be more easily inserted into an interbody space (for example, an interbody space that has collapsed, or any other suitable interbody space). In some embodiments, the implant further has a deployed configuration in which the height of the implant is greater than the height of the implant in the insertion configuration. Accordingly, in some cases, the implant is configured to expand when it is toggled (or otherwise adjusted) from the insertion configuration to the deployed configuration thus allowing it to be inserted more easily, yet to still adopt the proper height to provide the needed spacing and support for the adjacent vertebral bodies 20. In some embodiments, the implant further has a deployed configuration in which the height of the implant is less than the height of the implant in the insertion configuration; that is to say that the deployed configuration can be force limited, and so it is possible that when the implant is toggled from the insertion configuration to the deployed configuration, the implant could shrink in height if the loads within the space are too high for the implant to grow in height. The interbody spacer implant can have any suitable components or characteristics that allow it to toggle (or otherwise adjust) from an insertion configuration to a deployed configuration (i.e., to be deployed), as discussed in more detail below.

Where the implant 30 is configured to expand, it can be configured to expand any suitable amount. For example, according to some embodiments, a height of one or more portions of the implant (or a height of the implant overall) is configured to expand (e.g., from an insertion configuration to a deployed configuration) by anywhere from 1% to 500%, or any subrange thereof (e.g., between 1% and 100%, 5% and 90%, 10% and 85%, 20% and 80%, 25% and 75%, or any other suitable subrange).

According to some embodiments, the implant 30 is configured to conform to one or more bony endplates 24. Thus, in some cases, the implant is configured to eliminate one or more gaps between a surface of the implant and a bony endplate, provide a desired level of support across an uneven topology (or multiple different kinds of topologies) of a bony endplate, adopt a surface configuration that corresponds to the topology of a bony endplate, or otherwise conform to a bony endplate to avoid hyperloading or hypoloading. In some cases, the implant is configured to conform (e.g., adopt a conforming configuration) as it is deployed. Thus, some embodiments of the deployed configuration are also configured to conform to the topology of one or more bony endplates. In this regard, the interbody spacer implant can have any suitable components or characteristics that allow it to conform to one or more bony endplates.

Where the implant 30 is configured to conform to one or more bony endplates 24, the implant can conform to any suitable degree. For example, in some embodiments, a first portion of the implant expands substantially more than a second portion of the implant when the implant is deployed. In some cases, a first portion of the implant is configured to expand up to 200× more or less than a second portion of the implant when the implant is deployed (e.g., 150×, 100×, 75×, 50×, 25×, 10×, 5×, 2×, or any other suitable difference in expansion). In this regard, it is noted that, in some cases, the need for the most expansion is nearest to the center of the vertebral endplate. Accordingly, in some embodiments, a portion of the implant nearest its center is configured to expand more. Alternatively, in some embodiments, a bone defect may require an implant to be configured to expand most in the area of the bone defect. Thus, some embodiments of the described implant are tailored to the specific characteristics of one or more bones that are to be adjacent to the implant once it is properly seated in the patient.

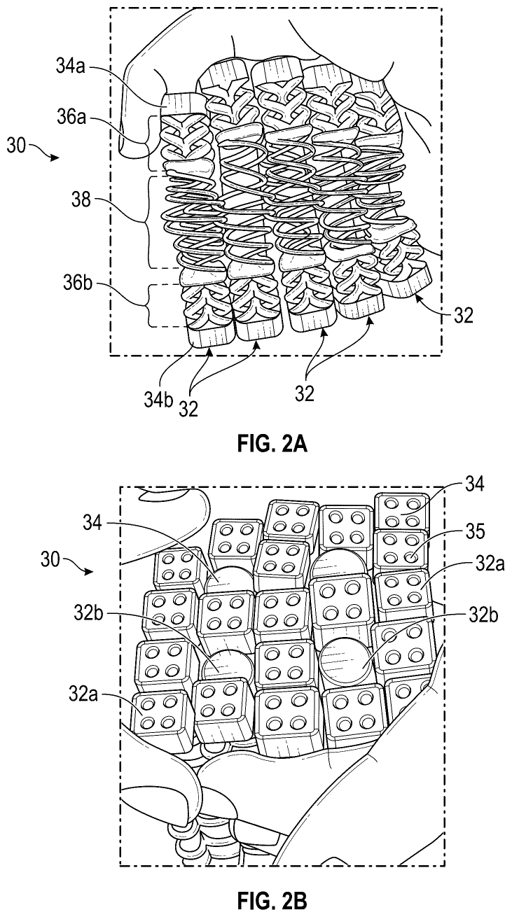

As mentioned above, the implant 30 can be configured to expand and conform in any suitable manner, but some embodiments of the implant include a plurality of components, which can include expanding components and fixed components (and any other type of components). These components can include any suitable components, such as one or more sheets, walls, rods, springs, frames, blocks, filaments, strands, coils, scaffolds, waveforms, or other components. For example, in some embodiments the components include one or more columns 32 configured to extend between a first bony endplate 24 of a first vertebral body 20 and a second bony endplate 24 of a second vertebral body 20. Columns (e.g., generally columnar components, in accordance with some embodiments, or any other suitable structures) may be particularly useful for some embodiments of the instant systems and methods. Accordingly, this disclosure generally describes the characteristics of the components with respect to columns. That said, for the purposes of this disclosure, references to columns can be substituted for references to other components, such as cubic, disc-like, sheet-like, prismatic, elliptical, elongated, conical, frustoconical, columnar, polygonal, symmetrical, asymmetrical, wave-like, strand-like, arch-like, or otherwise-shaped components, and the features described with respect to columns can be applied to such otherwise-shaped components. By way of non-limiting illustration, FIGS. 2A and 2B show some embodiments of the implant 30 having a plurality of columns 32, including one or more expanding columns 32a and fixed columns 32b.

The implant 30 can include any suitable number of columns 32 (or other components), such as anywhere from 2 to 1,000, or any subrange thereof (e.g., 5-50, 8-30, 10-20, 11-13, or any other suitable number).

Where the implant 30 includes one or more columns 32, the columns can be formed of or otherwise include any suitable material or materials. For example, some embodiments include one or more types of metal (e.g., titanium, titanium alloy, steel (e.g., stainless steel or other steel), tantalum, cobalt, chromium, magnesium, platinum, copper, aluminum, silicon nitride, ceramic, or any other suitable biocompatible metal or metal alloy), polymer material (e.g., polyethylene, polyamides, polyurethane, polycarbonate, polypropylene, polystyrene, polyvinyl chloride (PVC), acrylonitrile butadiene styrene (ABS), polylactic acid (PLA), polyetheretherketone (PEEK), polyaryletherketone (PEAK), or any other biocompatible polymer material), glass, plastic, carbon fiber, wood, fabric, ceramic, fiberglass, or any other material. By way of non-limiting illustration, some embodiments include titanium or one or more other metal materials, as such material selection can impart desirable strength characteristics to the implant. Moreover, some embodiments of the columns comprise silicon nitride.

Where the implant 30 comprises more than one column 32, the various columns can all comprise the same, different, or any suitable combination of material or materials. Indeed, in some embodiments, a first column is formed of or otherwise includes a first material or combination of materials, and a second column is formed of or otherwise includes a second material or combination of materials. In some cases, the first material or combination of materials is the same as the second material or combination of materials, but in some cases it is different. Similarly, a third column can include a third material (or combination of materials), which can be the same as or a different material from the first or second materials (or combinations), as can a fourth column, a fifth column, and any other suitable number of additional columns. Moreover, in accordance with some embodiments, selecting materials of varying moduli can enhance the specific function of each specific column (e.g., fixed columns 32b could be of a stiffer material and expanding columns 32a could be of a more flexible material). Additionally, in some embodiments, one or more materials can be selected for percent elongation at yield to enhance any suitable type of performance, such as fatigue resistance.

The columns 32 can also include any suitable configuration allowing them to load against (e.g., apply pressure, directly or indirectly, to) a spinal segment endplate 24. In some embodiments, the configuration of the columns (alone or in combination) allows the implant 30 (or one or more portions thereof) to conform to a variety of endplate topologies and to place a satisfactory load on a variety of endplates (without hypoloading or hyperloading the endplate or portions thereof) despite topological variations of such endplates. By way of example, some embodiments of the columns include one or more fixed components, stiff components, solid components, hollow components, expanding components, porous regions, frame regions, spring regions, resilient regions, rigid regions, fibrous regions, filamentous regions, foam regions, spongy regions, inflatable regions, flexures, grids, seal regions, cushion regions, sheets, curves, lattices, scaffolds, honeycombs, coil packs, or any other suitable configuration of one or more features that are configured to impart particular stiffnesses, strengths, or any other suitable attributes to the columns. Some examples of specific configurations of some suitable columns are provided in a later portion of this description below.

The columns 32 of the implant 30 can have any suitable stiffnesses for conforming to an endplate 24 and exerting a suitable load on the endplate. According to some embodiments, one or more of the columns is configured to have a stiffness between 10 MPa and 300 GPa (or any subrange thereof), although in many cases a stiffness closer to the range of between 100 MPa and 1.2 GPa (or any subrange thereof) is more desirable. For example, in some embodiments a column has a stiffness of between 100 MPa and 2 GPa, 200 MPa and 600 MPa, 200 MPa and 1.5 GPa, 300 MPa and 1.2 GPa, 500 MPa and 850 MPa, or any other subrange between 10 MPa and 100 GPa.

It should be noted that, in accordance with some embodiments, a stiffness of a column 32 during expansion or deployment can include a tradeoff between expansion height, desired force exerted on the implant 30 to obtain the desired distraction (in some cases, approximately 400 N total), and the maximum force to be exerted on the endplate 24 per unit area (in some cases, 4-8 MPa). Thus, in some embodiments, as the implant grows in footprint, the desired expansion stiffness (per unit area) can decrease.

In some embodiments, the implant 30 is configured to have a different stiffness while the implant is being deployed (e.g., while it is transitioning from an insertion configuration to a deployed configuration) than when it is in the deployed configuration. In some cases, the stiffness of the implant while it is being deployed is between 5% and 250% less than the stiffness of the implant in the deployed configuration (or any subrange thereof, such as between 10% and 200% less, 25% and 150% less, 30% and 100% less, 40% and 60% less, or any other subrange). In accordance with some embodiments, the stiffness of the implant when it is being deployed is below or near the fracture pressure of bone, while the stiffness of the implant when fixed is (in some embodiments) below 1.2 GPa.

As discussed in additional detail below, some embodiments include multiple different types of columns 32, such as an expanding column 32a and a fixed column 32b. In some cases, a first column (which can be an expanding column, a fixed column, or any other suitable type of column) has a first stiffness (e.g., any of the stiffnesses discussed above), and a second column (which can also be an expanding column, a fixed column, or any other suitable type of column) has a second stiffness (e.g., any of the stiffnesses discussed above, which can be the same or different from the first stiffness). In some embodiments, a combination of the first stiffness with the second stiffness results in a particular desired stiffness provided by the implant as a whole (e.g., any of the stiffnesses set forth above). By way of non-limiting illustration, in some embodiments, each of the first stiffness and the second stiffness is between 100 MPa and 1.2 GPa (or any subrange thereof). As another example, some embodiments of the first stiffness and the second stiffness combine to equal a combined stiffness of between 100 MPa and 1.2 GPa (or any subrange thereof). Some embodiments include additional columns with the same or different stiffnesses (or combinations of stiffnesses). In some embodiments, the expanding column is configured to have a stiffness that is tailored to its contact area so that the resultant contact pressure is less than the fracture pressure of the bone. In this regard, the exact values can be a design choice and can be enabled by this described geometry.

In some embodiments, the stiffness of the first column 32 (e.g., an expanding column 32a) is different from the stiffness of the second column (e.g., a fixed column 32b). For example, in some embodiments, the first stiffness is less than the second stiffness. That said, even though in some embodiments it is generally advantageous for the first stiffness to be less than the second stiffness, the first stiffness of some embodiments is more than the second stiffness. In some embodiments, the first stiffness is up to 99.9 GPa less (or more, in some embodiments) than the second stiffness. For example, in some cases the first stiffness is up to approximately 90 GPa, 75 GPa, 50 GPa, 25 GPa, 10 GPa, 5 GPa, 3 GPa, 2 GPa, 1 GPa, 900 MPa, 800 MPa, 750 MPa, 700 MPa, 650 MPa, 600 MPa, 550 MPa, 500 MPa, 450 MPa, 400 MPa, 350 MPa, 300 MPa, 250 MPa, 200 MPa, 150 MPa, 100 MPa, 50 MPa, 10 MPa, or any other suitable amount less (or more) than the second stiffness. In some embodiments, the stiffness differential between the first stiffness and the second stiffness falls within a range, such as a range including any two of the foregoing stiffnesses or any other range between 1 MPa and 99.9 GPa. By way of non-limiting illustration, in some embodiments, the second stiffness is between 5 MPa and 1.2 GPa stiffer than the first stiffness (or vice versa). Moreover, in some embodiments, the second stiffness is between 10 MPa and 200 MPa stiffer than the first stiffness (or vice versa).

In some embodiments, by including multiple columns 32, a wide variety of endplate 24 topologies can be accommodated with a single implant 30. Indeed, in some embodiments, the implant causes a desirable load to be exerted across a large percentage of a surface area of an endplate (e.g., more than 50%, 60%, 70%, 80%, 90%, 95%, or 99%, or even up to 100% of the surface area of the endplate) notwithstanding variations in the topology of the endplate or variations between different endplates. In some cases, the desirable load to be exerted across the endplate is between 50 MPa and 5 GPa (or any subrange thereof). For example, in some embodiments, the implant is configured to provide a load of between 300 MPa and 1.2 GPa (±150 MPa) across a large percentage of a variety of different endplates with different topologies (e.g., at least 90%, 95%, 99%, or more of naturally occurring human endplates). Moreover, in some embodiments, the implant is configured to provide a load of between 100 MPa and 4.1 GPa (±100 MPa) across a large percentage of a variety of different endplates with different topologies.

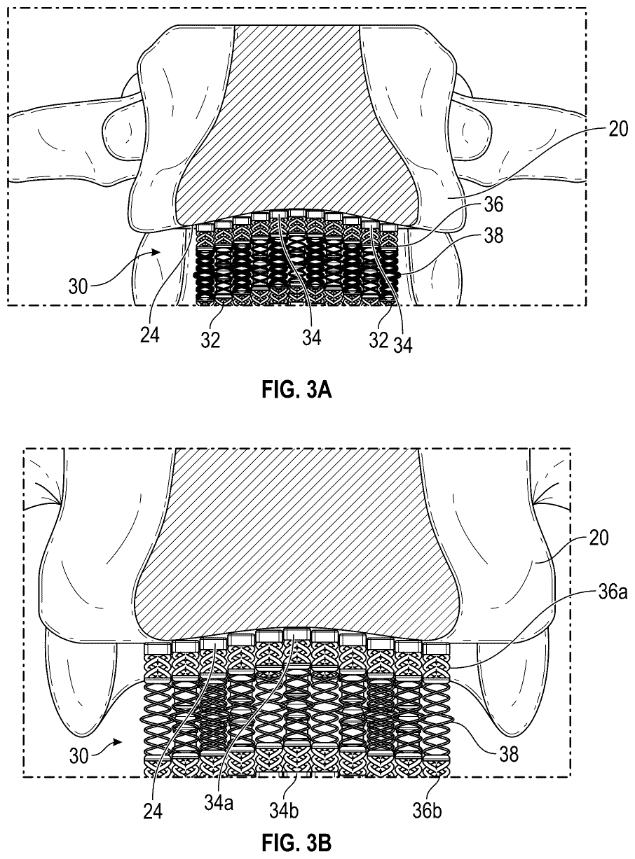

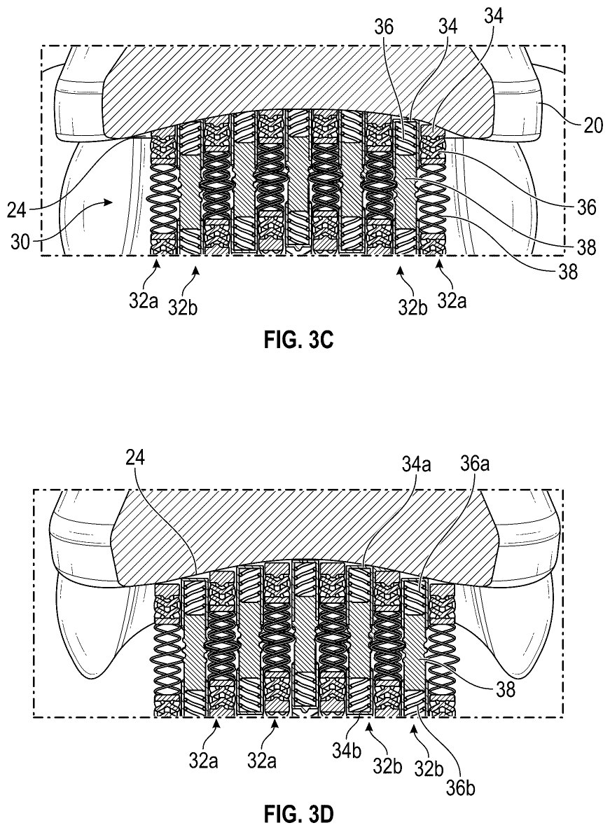

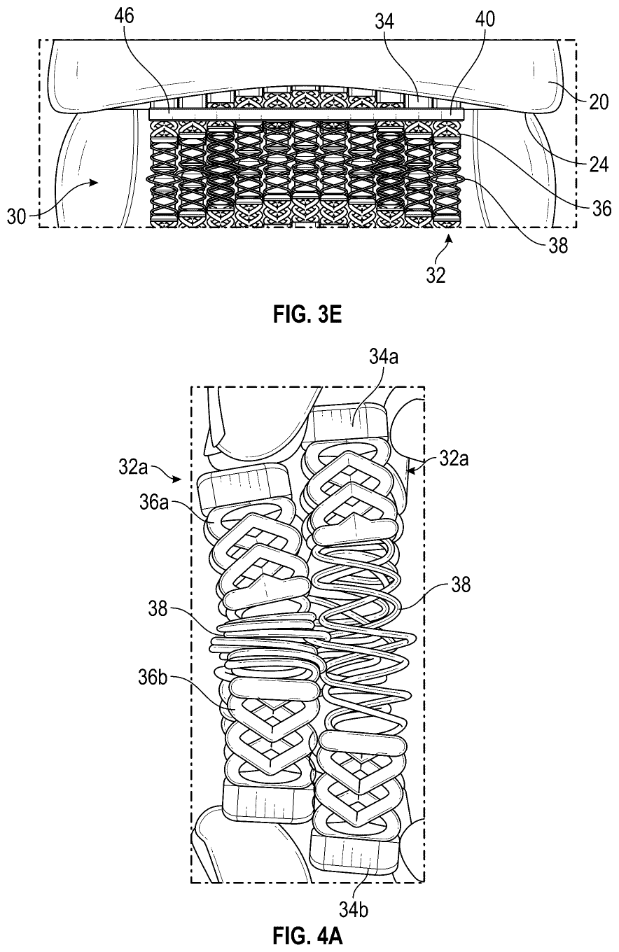

According to some embodiments, the implant 30 is tailored to conform not only to different depths of endplate 24 topology, but also to different regions of the endplate (e.g., the central region, the posterior region, the lateral region, the anterior region, or another region). In this regard, endplates generally vary in stiffness, strength (e.g., load-bearing capacity), and shape across their various regions. In some embodiments, the implant is tailored to meet specific needs depending on these natural variations. For example, in some embodiments, the implant includes one or more regions that correspond with one or more regions of an endplate. In this regard, some embodiments of the implant include one or more surfaces (e.g., a rostral surface, a caudal surface, or any other suitable surface) that include one or more of a central region (e.g., corresponding with the central region of an endplate), a posterior region (e.g., corresponding with the posterior region of an endplate), a left lateral region (e.g., corresponding with the left lateral region of an endplate), a right region (e.g., corresponding with the right lateral region of an endplate), an anterior region (e.g., corresponding with the anterior region of an endplate), or any other suitable region. By way of non-limiting illustration, FIGS. 3A-3E show an expandable and conforming implant 30 with multiple columns 32 conforming to an endplate 24 of a vertebral body 20.

In some embodiments, one or more regions of the implant 30 are modified to have one or more differences, such as a different stiffness, a different size, a different shape, a different contact area, a different pore density, a different material composition, different structure, or another different attribute. By way of non-limiting example, in some embodiments, at least one of a posterior region, a left region, a right region, an anterior region, a central region, or another region of the implant has a greater (or lesser) stiffness than at least one other region of the implant. Where this stiffness is greater, this can allow a greater load to be transferred to the corresponding region of the endplate. In this regard, the central region of the bony endplate can sometimes receive the least amount of load from an implant, as the depth of the topology tends to be greatest at the central region (so the deflection of the corresponding central region of the implant is least, compared to the other regions). Thus, to compensate, some embodiments of the central region of the implant have increased stiffness, increased length (or maximum length), or any other suitable difference, thereby increasing the load applied to the central region of the endplate (and causing a more uniform load to be applied to the endplate overall).

In accordance with the foregoing, some embodiments of the implant 30 have a surface (e.g., formed of multiple ends 34 of columns 32) that is adapted across various portions of the implant. In this regard, differences included in the surface (if any) can be arranged in any suitable manner. In some embodiments, the differences within the surface closely align with the regions of the bony endplate 24. In some embodiments, the surface includes differences within the various regions. For example, some embodiments of the surface include a gradient, with a particular difference (e.g., stiffness) increasing or decreasing medially to laterally, left to right, anterior to posterior, central to exterior, or in another configuration throughout all or a portion of the implant. Some embodiments include differences within the surface arranged in a custom-tailored manner or in another specific arrangement (e.g., in a particular geometric pattern). It is noted that the vertebral endplate is often stiffest at the lateral edges, with decreasing stiffness toward the center and also somewhat toward the ventral aspect. In any case, tailoring the stiffness of the implant to best match the bony endplate can, in some cases, ameliorate risk of bone damage.

In some embodiments, the implant 30 is configured to conform to a single endplate 24, but some embodiments of the implant are configured to conform to multiple endplates or to fill one or more voids in a vertebral body. For example, some embodiments of the implant include multiple surfaces, such as a rostral surface and an opposing caudal surface, a ventral surface and an opposing dorsal surface, or a right surface with an opposing left surface. In this regard, some embodiments of the implant are “double-sided”, with a rostral conforming region on a rostral surface of the implant (configured to conform with a caudal-facing endplate above the implant) and a caudal conforming region on a caudal surface of the implant (configured to conform with a rostral-facing end plate below the implant). As with the various regions (or portions of regions) within a surface (as discussed above), where multiple conforming regions are included, the conforming regions can differ from each other in any suitable manner (including in stiffness, size, shape, contact area, design, pore density, material composition, structure, or any other suitable attributes). In any case, any of the features and characteristics discussed herein can be applied to one or both surfaces or conforming regions.

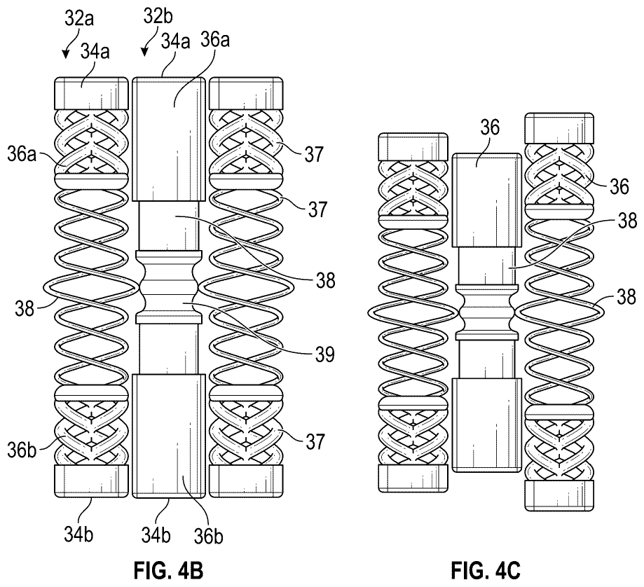

According to some embodiments, the implant 30 is configured to be “sandwiched” between two vertebrae 20. Accordingly, in some cases, when a rostral endplate applies load to an implant endplate (e.g., a rostral surface), the load is applied on the other side as well (e.g., at the caudal surface loaded against a caudal endplate). Yet, the contact pressure between implant and bone can (in accordance with some embodiments) be made to vary across the contact region to match the natural or exceptional variation in bone density.

Where the implant 30 includes multiple columns 32, the columns can include any suitable component and have any suitable configuration that allows them to accomplish their intended purposes. As mentioned above, some embodiments include multiple different types of columns, such as one or more expanding columns 32a, one or more fixed columns 32b, or one or more additional types of columns. In some cases, all columns (including columns of different types) share one or more characteristics, and in some cases, columns of different types have different sets of characteristics. In accordance with the foregoing, descriptions of the various different kinds of columns are provided below, but it is noted that (in accordance with some embodiments) an expanding column can have or lack any characteristic described with respect to a fixed column and vice versa.

Where the implant 30 includes one or more expanding columns 32a, the expanding columns can include any component or have any configuration allowing them to expand or to effectuate an expansion of the implant. In some embodiments, the expanding columns have multiple regions that can differ from each other in stiffness (e.g., in accordance with any of the stiffnesses discussed above), porosity, strength, material, design, or any other characteristics. For example, some embodiments of the expanding columns include one or more outer regions 36 and one or more inner regions 38 (in this case, the term “outer” can mean disposed toward a cranial or caudal surface or end 34 of the column, and the term “inner” can mean disposed at or near a plane positioned halfway between the caudal and cranial surfaces or away from the ends of the column). In some cases, by having different regions of different stiffnesses, the implant is able to have a different effective stiffness while it is being shifted from the insertion configuration to the deployed configuration than when it is in the deployed configuration for everyday use. By way of non-limiting illustration, FIG. 4A shows expanding columns 32a, each having a first outer region 36a disposed proximate to a first end 34a and a second outer region 36b disposed proximate to a second end 34b, with an inner region 38 disposed between the first outer region and the second outer region.

Similarly, Where the implant 30 includes one or more fixed columns 32b, the fixed columns can include any component or have any configuration allowing them to provide support for the implant (e.g., for the expanding columns 32a). In some embodiments, the fixed columns have multiple regions that can differ from each other in stiffness (e.g., in accordance with any of the stiffnesses discussed above), porosity, strength, material, design, or any other characteristics. As with the expanding columns, some embodiments of the fixed columns include one or more outer regions 36 and one or more inner regions 38. By way of non-limiting illustration, FIGS. 4B and 4C show fixed columns 32b alongside expanding columns 32a, each having a first outer region 36a disposed proximate to a first end 34a and a second outer region 36b disposed proximate to a second end 34b, with an inner region 38 disposed between the first outer region and the second outer region.

Although some embodiments of the implant 30 comprise only a single layer of columns 32 (e.g., as shown in FIGS. 4A-4C), in some embodiments, two or more columns are stacked (e.g., arranged in series) in the implant. In such embodiments, any suitable number of columns can be stacked end to end, including 2, 3, 4, 5, 6, or more. Although in some embodiments in which two or more columns are stacked end to end, the columns are not physically locked together, in some other embodiments, two or more columns are physically coupled together, end to end. In such embodiments, the columns can be coupled together in any suitable manner, including through the use of one or more frictional couplings, set screws, mechanical couplings, fasteners, magnets, adhesives, catches, couplers, welds, claps, integrally formed couplings, or in any other suitable manner.

In some embodiments, an end 34 of a column 32 (e.g., a first end 34a or a second end 34b of an expanding column 32a or a fixed column 32b) is configured to contact a bony endplate 24. Accordingly, the end of the column can be configured in any suitable manner to place a load on the endplate, support the endplate, stimulate bone growth, or otherwise positively affect the interaction between the implant 30 and the endplate. For example, the end can have any suitable shape, including circular, semi-circular, triangular, square, rectangular, trapezoidal, pentagonal, hexagonal, star-shaped, T-shaped, polygonal, symmetrical, asymmetrical, or any other regular or irregular shape. By way of non-limiting illustration, FIG. 2B shows columns with various ends 34, including fixed columns 32b having circular ends 34 and expanding columns 32a having square ends 34 with radiused or rounded corners.

In some embodiments, one or more of the ends 34 include one or more surface features 35 for stimulating bone growth or otherwise impacting the ends' interaction with the endplate 24. The surface features can include any suitable surface features, including one or more pores, ridges, grooves, beads, bumps, divots, microstructures, textures, stimulants, hormones, or other features for stimulating bone growth. By way of non-limiting illustration, FIG. 2B shows ends 34 with surface features 35 in the form of pores (which can have a porosity, as described in additional detail in a later portion of this disclosure). In some embodiments, any suitable number of the ends are optionally configured to pierce or extend into the endplate to improve fixation.

In embodiments with columns 32 that include one or more outer regions 36, the outer regions can have any suitable component or configuration that allows them to provide adequate implant support. For example, in some embodiments, the outer region is substantially solid, rigid, and otherwise structurally configured to support the load placed on it by the endplate 24. In some embodiments, the outer region includes a porous structure 37 (as discussed in more detail in a later portion of this disclosure).

In embodiments with columns 32 that include one or more inner regions 38, the inner regions can have any suitable component or configuration that allows them to provide adequate implant support or, in the case of expanding columns 32a, to expand to increase the height of the column or to contract to place the implant 30 in the insertion configuration. For example, in some embodiments, the inner region is substantially solid and rigid (e.g., in the case of fixed columns 32b), and otherwise structurally configured to support the load placed on it by the endplate 24. In some embodiments, the inner region includes one or more porous structures 37 (as discussed in more detail in a later portion of this disclosure). Where the outer region 36 and the inner region 38 each include one or more porous structures 37, the porous structure of the inner region 38 can have different characteristics than the porous structure of the outer region (e.g., a different stiffness, a different porosity, a different strand layout, or another difference). Similarly, the inner region can differ from the outer region in any other respect. For example, some embodiments of the inner region have a diameter that is smaller (or larger) than a diameter of the outer region. Some embodiments of the inner region have a different shape, different contours, a different stiffness, different design, or any other suitable different characteristics. In some embodiments, the inner region includes one or more recesses 39 (which, in some cases, are configured to accommodate one or more strands of a porous region of an adjacent column). In some embodiments, one or more of the fixed columns are configured with, or define one or more pores, or include one or more features to decrease their stiffness and to increase vascularization.

By way of non-limiting illustration, FIGS. 4B and 4C show some embodiments of expanding columns 32a and fixed columns 32b, where the fixed columns have outer regions 36 and inner regions 38 that are each substantially solid and configured to support the load placed on the implant by the endplates. In these illustrated embodiments, the inner region 38 has a smaller diameter than the outer region 36, and it is further contoured, having a recess 39 which can accommodate a strand of a porous structure 37 of a neighboring column. The expanding columns 32a, as illustrated, also have outer regions 36 and inner regions 38, but each of these regions includes a porous structure 37 (the porous structure having thicker strands, and thereby a greater stiffness, at the outer regions). Accordingly, some embodiments of the expanding columns 32a are configured to expand (e.g., from a position in which they have a height approximately equal to a height of the fixed column 32b, as shown in FIG. 4B, to a position in which they have a height greater than the height of the fixed column 32b, as shown in FIG. 4C).

Although the columns 32 and their regions can have any suitable dimensions, in some cases a column (e.g., a fixed column 32b) has a height of approximately 10 mm±8 mm (or any subrange between 2 mm and 18 mm). In some embodiments, an outer region 36 (e.g., a first outer region 36a or a second outer region 36b) of a column (whether a fixed column or an expanding column 32a) has a height of approximately 4 mm±3 mm (or any subrange thereof).

According to some embodiments, the height of one or more columns 32 (including fixed columns 32b, or a deployed or pre-deployed height of expanding columns 32a) is selectively adjustable. In this regard, a column can be configured to be selectively adjustable in any suitable manner, such as by using one or more expandable components, flexible components, telescoping components, threaded components, or any other suitable height-adjustable components of any kind. For example, in some embodiments, the outer regions 36 of a column 32 can include a threaded connection with the inner region 38, such that twisting an outer region with respect to the inner region causes the outer region to move up or down with respect to the inner region, thereby lengthening or shortening the column. When such a system is used in connection with a fixed column, this can (in some embodiments) be useful for adjusting the height of the implant in the insertion configuration. In some cases, the fixed columns have a fixed height, and fixed columns of the desirable height are preselected prior to formation of the implant. Moreover, in some embodiments, a fixed column is expandable (or can be lengthened) such that an expansion of the fixed column is configured to expand less than an expansion of an expanding column.

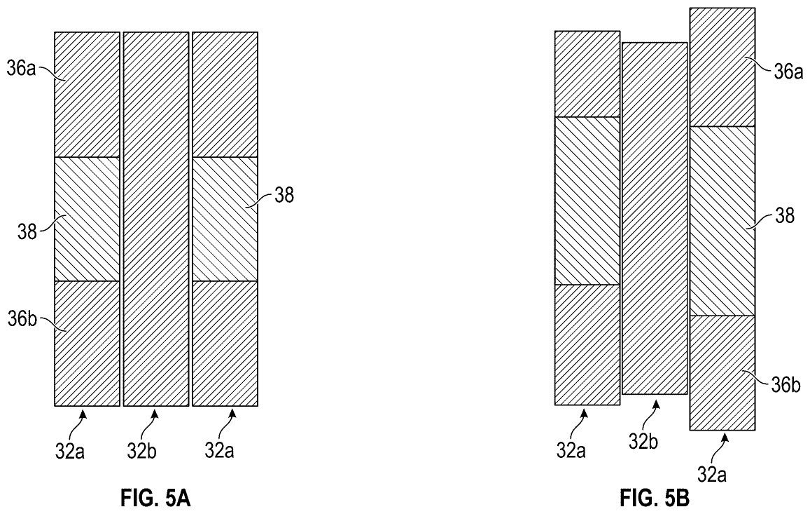

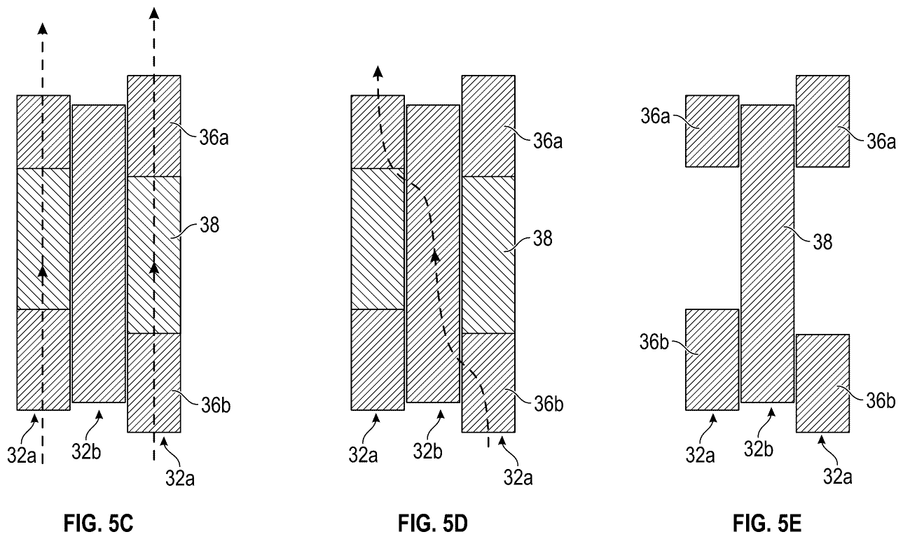

Although deployment of the implant 30 can be accomplished in any suitable manner that causes the overall height of the implant to increase and one or more surfaces (e.g., the caudal surface and the cranial surface) to conform to the respective endplates, according to some embodiments, when the implant 30 is toggled from an insertion configuration to a deployed configuration, the height of the expanding columns 32a increases, while the height of the fixed columns 32b remains the same (e.g., at a pre-insertion height, which, as discussed above, can be preadjusted in some embodiments). For example, FIG. 5A shows an embodiment of a fixed column 32b surrounded by expanding columns 32a, as may be found in an implant in an insertion configuration. FIG. 5B shows an embodiment of the same columns, only in a deployed configuration, in which the inner region 38 of the expanding columns has been allowed to expand, thereby increasing the height of the expanding columns and the overall height of the implant, while the fixed column remains at a constant height.

Where the implant 30 includes one or more expanding columns 32a, the expanding columns can be configured to expand any suitable amount. For example, in some cases, an expanding column is configured to expand from a height of 10 mm (±8 mm) in an unexpanded state to a height of 18 mm (±10 mm) in an expanded state (including any subranges that fall within the foregoing ranges).

According to some embodiments, when the expanding columns 32a are allowed (or caused) to expand, they press against the bony endplates 24 of the vertebral bodies 20 on either side. Accordingly, the load on the endplates is (at least initially) borne by the expanding columns. In some embodiments, the expanding columns are configured to apply a proper load to the implants when they transition from the insertion configuration to the deployed configuration. As discussed above, in some cases, it is desirable for the load to be less during expansion than during everyday loading. Accordingly, some embodiments are configured to have a lesser stiffness during expansion. Thus, generally speaking, if the implant is configured to have a particular stiffness during expansion, the stiffness of any particular expanding column can be such that the stiffnesses of all the expanding columns taken together result in the desired implant stiffness.

In accordance with the foregoing, some embodiments of deploying the implant 30 include expanding the expanding columns 32a (or allowing them to expand) until the expanding columns press against the opposing bony endplates 24 with a desired force (e.g., a desired deployed force in particular, a force that avoids hyperloading or hypoloading the bony endplates—is achieved). In some embodiments, this is done by manually expanding the expanding columns (e.g., through inflation of a balloon, or otherwise, as described in more detail below). In some embodiments, this is done automatically (e.g., a stiffness of the expanding column, or a stiffness of particular structure thereof (such as the inner region 38), is configured to apply the desired amount of force. For example, if the expanding column includes a spring-like element (e.g., a strand, as discussed below), the spring-like element can be configured to expand until the desired load force is reached. By way of non-limiting illustration, FIG. 5C shows a load (represented by the dashed arrowed lines) being directed through the expanding columns 32a bordering a fixed column 32b.

According to some embodiments, the implant 30 is configured such that the load can be selectively rerouted, transmitted, or otherwise transferred to pass through one or more fixed columns 32b instead of passing entirely through expanding columns 32a after it is deployed. While this can be accomplished in any suitable manner, in some embodiments this is done by coupling the outer regions 36 of the expanding columns to the fixed columns. With the outer regions of the expanding columns coupled to the fixed columns, the load path can pass through the outer regions of the expanding columns and through the fixed columns without needing to pass through the inner regions of the expanding columns (thereby removing the need to continue to rely on the expanding components of the inner regions after the expansion has been effectuated). By way of non-limiting illustration, FIG. 5D shows a pair of expanding columns 32a on either side of a fixed column 32b, with a load path (illustrated by the dashed arrowed line) traveling through a first outer region 36a of a first expanding column 32a a fixed column 32b and a second outer region 36b of a second expanding column 32a.

According to some embodiments, after the outer portions 36 of the expanding columns 32a are coupled to the fixed columns 32b, the inner portions 38 of the expanding columns can be removed. In some cases, the inner portions remain in place, but the load is largely or wholly rerouted away from the inner portions. Accordingly, the stiffness of the implant 30 is transitioned from the stiffness of the expanding columns to the stiffness of the fixed columns, thus allowing for a different stiffness in the deployed configuration than while the implant is being deployed. Furthermore, in some cases, removing the inner portions or rerouting the load can remove variation that could otherwise be caused by the inner portions. For example, certain types of expanding components (e.g., balloons, springs) are very useful for expansion, but they may not be as stable as other components (e.g., having a lesser stiffness can potentially fail to provide the necessary support). That said, some embodiments are configured such that the expanding components continue to bear all or a substantial portion of the load throughout the life of the implant, so rerouting the load is not necessary in every embodiment.

In some embodiments, the inner portions 38 of the expanding columns (e.g., the expanding components) are selectively inserted only as necessary to deploy the implant 30. For example, in some embodiments, the expanding component includes an inflatable element (e.g., a balloon). An example use of such an element is as follows: the expanding element is inserted into the implant after the implant has been inserted into an intervertebral space, and then the expanding component is used to deploy the implant by causing the expanding columns' height to increase, and then the expanding columns are coupled to the fixed columns, and then the expanding element is removed. By way of non-limiting illustration, FIG. 5E shows a fixed column 32b with outer regions 36 of expanding columns 32a fixed thereto, thereby remaining in the expanded configuration notwithstanding the absence of inner regions (not pictured) of the expanding columns.

To summarize some of the foregoing concepts, FIGS. 5A-5E show an example how a deployment of the implant might work: first, the implant is in an insertion configuration, and the expanding columns 32a are unexpanded (e.g., they are the same height as the fixed columns 32b, as shown in FIG. 5A); next, the expanding columns are expanded (e.g., through expansion of the inner regions 38, as shown in FIG. 5B), thereby taking on the load from the endplates (as shown in FIG. 5C) and distracting the disc space; next, the outer regions 36 of the expanding columns are coupled to the fixed columns, such that the load is at least partially rerouted through the fixed columns (as shown in FIG. 5D), effectively strengthening and stiffening the implant to the desired deployed configuration stiffness; next, the inner regions of the expanding columns are removed (or made obsolete), such that the expanded implant bears the load through the fixed columns and the outer regions of the expanding columns (as shown in FIG. 5E). Again, this is just an illustrative example, and variations are possible. For example, in some embodiments, the inner region of the expanding columns continues to bear all or part (e.g., 90%, 80%, 75%, 50%, 25%, 10%, or any other portion) of the load throughout the useful life of the implant.

Where the implant 30 includes one or more expanding columns 32a, the expanding columns can be configured to expand in any suitable manner. Some embodiments utilize an inflatable element, as discussed above (e.g., a specialized balloon configured to expand due to the pressure of a gas blown or pumped into the inflatable element). Some embodiments utilize a mechanical tool (e.g., to pry or otherwise force the first outer region 36a away from the second outer region 36b, thereby increasing the height of the expanding column). In some embodiments, the expanding element is integral to the structure of the expanding column. For example, in some embodiments, a spring-like element (or another element configured to expand under its own power in-situ) is used, which can be compressed to put the implant into the insertion configuration, and which naturally decompresses when allowed to due to the compression on the spring-like element.

In some embodiments that utilize a spring-like element, the spring-like element is configured to remain in an unexpanded (compressed) state until the implant 30 is deployed. While the implant can have any suitable configuration for preventing the expanding column 32a from expanding until the proper time, some embodiments of the expanding column are configured to selectively couple to one or more fixed columns 32b while in the unexpanded state. Accordingly, in some embodiments, the expanding column is configured to automatically expand when decoupled from the fixed column. In some embodiments, the same mechanism used to couple the expanding column to the fixed column when the expanded column is in the expanded state is used to couple the expanding column to the fixed column when the expanded column is in the unexpanded state. That said, in some embodiments, different coupling mechanisms are used for coupling in the expanded state than are used for coupling in the unexpanded state.

Where the expanding column 32a is configured to couple to the fixed column 32b (whether in the expanded state, in the unexpanded state, or both), the coupling can be effectuated in any suitable manner, but some embodiments use one or more couplers 40. Where a coupler is used, the coupler can include any suitable coupling device, such as one or more set screws, eyelets, magnets, hook-and-loop fasteners, adhesives, welds, interference fits, friction fits, tongue-and-groove connections, snaps, ties, pins, rods, bands, rivets, zip ties, or any other coupling mechanisms. In some embodiments, one or more couplers are used to couple other columns 32 together (e.g., a first fixed column to a second fixed column, or a first expanding column to a second expanding column).

In some embodiments with a coupler 40, the coupler includes one or more dovetails 42. Where a dovetail is used, the dovetail can include any suitable tongue-and-groove-type components formed on adjacent columns. For example, in some cases, a first column include a protrusion, and a second column includes a receptacle configured to receive the protrusion. In some embodiments, the dovetail is configured to allow for only a single degree of motion (e.g., in a vertical (caudal-cranial) direction.

In some embodiments with a coupler 40, the coupler includes one or more pins 44. As with the dovetail 42, the pin can include any component configured to restrain movement of adjacent columns 32 with respect to each other. In some cases, the pin is configured to form a friction or interference fit between columns. In some cases, the pin is configured to form a friction or interference fit utilizing a column's structure (e.g., by fitting into a porous structure 37 or a recess 39 of a column). Some embodiments of the pin are configured to restrain movement that would otherwise be allowed (for example, in some embodiments where a dovetail allows cranial-caudal motion, the pin restricts cranial-caudal motion-accordingly, the pin and the dovetail work together to prevent movement of the columns with respect to each other). According to some embodiments, the pin is configured to be selectively removed (and replaced, in some cases) to allow for easy decoupling and recoupling of adjacent columns to each other. Although the pin can have any suitable shape, in some embodiments, the pin is wedge shaped and can be inserted in any suitable direction in order to constrain the column, such as in a medial-lateral, anterior-posterior, or cranial-caudal direction. In some embodiments, the pin (or wedge) is configured to extend into the endplate, thus providing an additional method of securing the implant 30 to the bone.

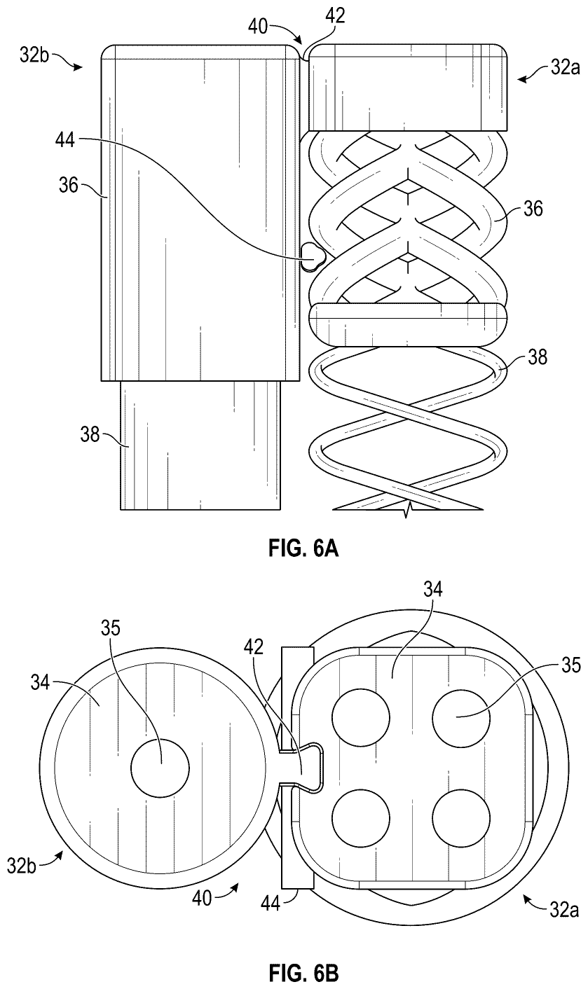

By way of non-limiting illustration, FIGS. 6A and 6B show an expanding column 32a coupled to a fixed column 32b via a coupler 40, where the coupler includes a dovetail 42 in the form of a protrusion and complementary receptacle allowing for movement only in a caudal-cranial direction, as well as a pin configured to interface with the outer region 36 of the expanding column and further configured to restrain movement in the caudal-cranial direction. Accordingly, when the pin is removed, the expanding column will be free to expand (which it will naturally do due to the compression of the spring-like structure of the inner region 38), thereby pushing the outer region cranially (upward).

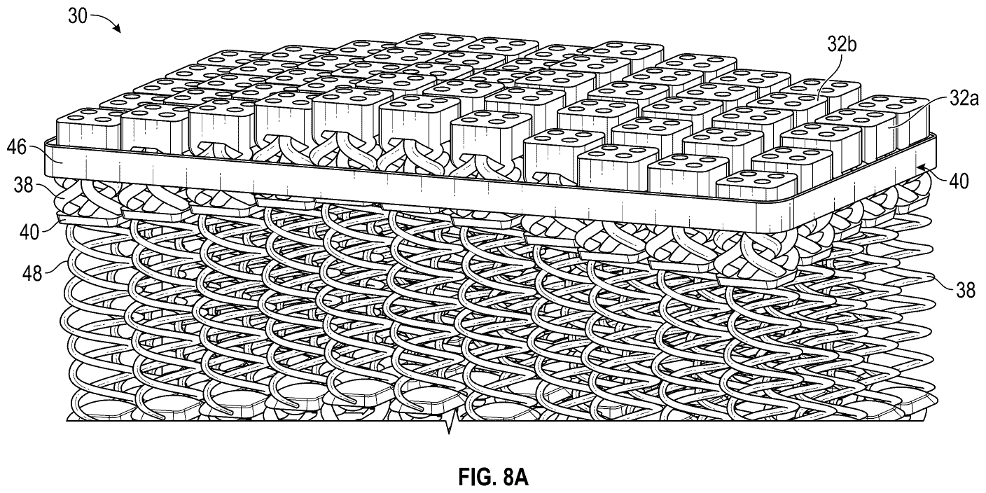

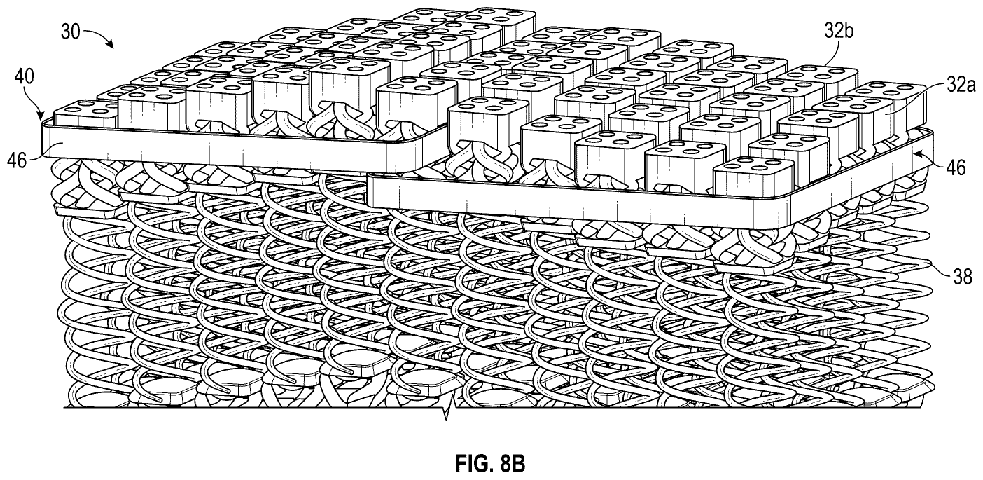

In some embodiments having the coupler 40, the coupler includes one or more bands 46. The band can include any component configured to hold one column 32 to another column (e.g., an expanding column 32a to another expanding column, to a fixed column 32b, or to any number of additional columns). In some embodiments, the band is configured to hold two or more columns together via friction (e.g., by forcing the columns together through a contracting force). The band can be made of any suitable material (e.g., elastic, rubber, metal (with a tight fit), or any other material configured to hold multiple components together by surrounding them (completely or partially) and pushing them together). By way of non-limiting illustration, FIG. 8A shows an implant 30 having a plurality of columns 32a, 32b coupled together by a coupler 40 that includes a band 46. Similarly, FIG. 8B shows an implant 30 with columns 32a, 32b coupled together via a coupler 40 that includes multiple bands 46. Any practical number of bands may be used.

In some embodiments that include a band 46, the band is configured to be selectively loosened and tightened. Accordingly, the band can be loosened to deploy the implant from the insertion configuration to the deployed configuration, allowing all expanding columns 32a to expand, and then the band can be tightened again to ensure that the columns are firmly coupled together (and allowing the fixed columns 32b to take at least part of the load transferred from the outer regions 36 of the expanding columns).

In some embodiments, the coupling is effectuated via one or more phase changes. For example, in some cases, the coupling is accomplished via a polymer melting under an inductive load, Nitinol or another compound undergoing a phase change, application of an adhesive, or another phase-change-related coupling.

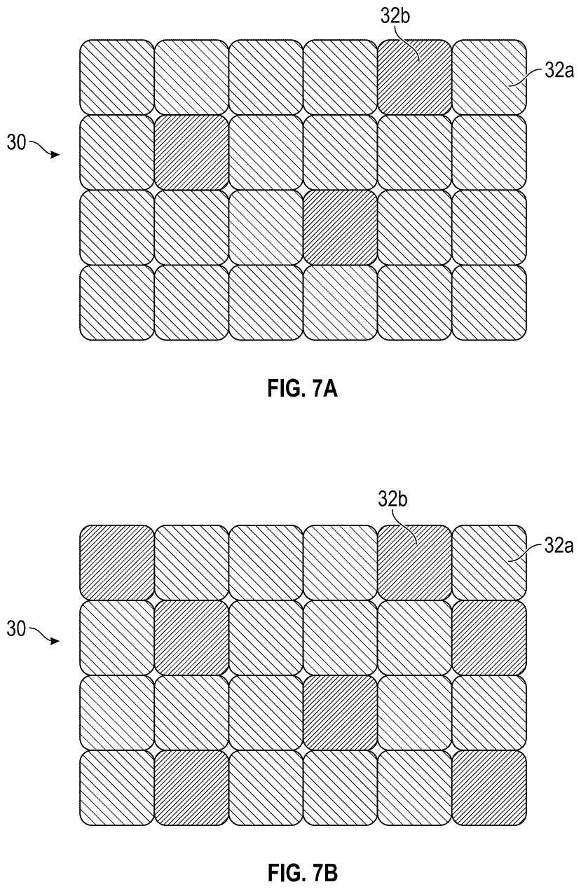

According to some embodiments, a retention force (e.g., due to partial application of a coupler 40 or otherwise) is used to limit or slow the expansion of the implant 30. In some embodiments, the retention force is applied in localized regions to encourage formation of curvature (e.g., in the spine, such as lordosis, kyphosis, or other curvature).

Where the implant 30 is configured to be deployed (e.g., toggled from the insertion configuration to the deployed configuration), the deployment can be activated in any suitable manner. For example, as discussed above, the deployment of some embodiments is configured to be activated by changing the configuration of a coupler 40 (e.g., loosening a band, removing a pin, twisting a portion of the coupler, decoupling a portion of the coupler, or otherwise changing the coupler's configuration). In some embodiments, deployment is activated in another way, such as by activating an actuator (e.g., via pressing a button, flipping a switch, turning a dial, or otherwise activating the actuator), inflating an inflatable element, or activating an anchor deployment mechanism. In some cases where an anchor deployment mechanism is used, the anchor deployment mechanism can be used to wedge a component between the sundry columns 32 and an outer ring, band 46, or any other suitable coupler to compress-lock (or otherwise couple) the columns to each other. In some such cases, actuating the anchor deployment mechanism actuates the expansion features. Thus, an anchor deployment mechanism is, in some cases, employed as a wedge between the band and the columns to selectively loosen and tighten (e.g., to effectuate and control deployment). Examples of suitable anchor deployment mechanisms are included in a later portion of this disclosure.

Where the implant 30 includes expanding columns 32a and fixed columns 32b, the columns can be arranged in any suitable manner. For example, the columns can be dispersed evenly, randomly, in a geometric pattern, in an optimized fashion as determined by patient-specific or spinal-segment-specific analysis, or in any other manner. In some embodiments, each expanding column is positioned adjacent to at least one fixed column. In some embodiments, each fixed column is positioned adjacent to at least one expanding column. In some embodiments, each expanding column is positioned adjacent to at least one other expanding column. In some embodiments, the columns alternate (like squares on a chess board), with each expanding column being positioned adjacent to only fixed columns, and vice versa. In some embodiments, all columns on an external perimeter of the implant are expanding columns. In some embodiments, all central columns are expanding columns. In some embodiments, the arrangement of expanding columns and fixed columns corresponds to (or complements) the natural endplate 24 organization. In some embodiments, each expanding column is adjacent to at least one fixed column or at least one expanding column that is itself adjacent to at least one fixed column. Further, although some embodiments are arranged generally in a square grid, other possible arrangements include a hexagonal grid, another type of tessellating or non-tessellating grid, with no specific grid, with a variable grid (e.g., with columns offset from each other instead of aligned edge-to-edge), in an arrangement that is tailored to one or more particular bones, in randomized arrangement, or in any other suitable manner. In short, many different arrangements are possible. By way of non-limiting illustration, FIGS. 7A and 7B show example dispersions of expanding columns 32a and fixed columns 32b within an implant 30.

Some embodiments of the implant 30 (e.g., any column 32 of the implant or any part thereof) can have any suitable characteristic that allows the implant to function as intended, including being planar, contoured, defining one or more openings, being porous, or having any other suitable characteristics. Indeed, some embodiments of the implant include one or more porous structures 37. In this regard, a properly configured porous structure can be highly beneficial in promoting healthy bone growth. Indeed, the cells generally responsible for bone growth, namely osteocytes and osteoblasts, typically work together to form bone as needed within the body. But these cells will typically only form bone under proper conditions, including when the cells experience proper loads and stresses, when a network of blood vessels is available to supply needed nutrients, and when gaps to be filled by bone are of a proper size. When proper conditions are not available, bone often cannot or will not grow. For example, when bone does not experience proper loading (e.g., due to hypoloading), it generally will not grow and can even be resorbed. Additionally, when gaps (i.e., pores) to be filled are too large, bone may not be able to bridge the gaps and often will not grow. Similarly, if pores are too small, bone growth may be impeded by the insufficient size. Thus, some embodiments of the implant employ a combination of a porous structure with proper loading (e.g., as a result of the conforming region) to stimulate improved bone growth.

Where embodiments include a porous structure 37, the porous structure can include any structure having gaps, spaces, air pockets, voids, cavities, hollows, or other pores capable of promoting bone growth (e.g., cortical bone growth, trabecular bone growth, or other bone growth).

According to some embodiments, the porous structure 37 includes one or more pluralities of pores. The pores can include any gaps, spaces, voids, air pockets, cavities, hollows, or other pores that are defined by the porous structure. For example, a hole in a strand qualifies as a pore in some embodiments, and a gap between multiple strands (or multiple portions of the same strand) qualifies as a pore in some embodiments.

In some embodiments, each pore of the plurality of pores has a pore size, or the plurality of pores has an average pore size, of between 10 μm and 3,000 μm or within any subrange thereof. For example, in some embodiments, the plurality of pores each have a pore size, or they have an average pore size, of between 100 μm and 1,000 μm, between 150 μm and 700 μm, between 150 μm and 650 μm, or any other subrange of between 10 μm and 3,000 μm. That said, a pore size of between approximately 150 μm and 650 μm (±100 μm) may be particularly useful for promoting bone growth in combination with the loads placed on bony endplates 24 as a result of the stiffnesses selected for the columns 32 of some embodiments of the implant 30 (as discussed above). In accordance with some embodiments, pores larger than 450 μm can allow for vascularization and pores smaller than 650 μm can allow for bone bridging. Additionally, in some embodiments, areas or pores larger than 650 μm can be selectively placed to increase radiolucency.

The plurality of pores can include any number of pores (e.g., between 2 and 50,000, or any subrange thereof, such as 100±50, 1000±500, 1500±750, 10,000±5000, or any other desired number of pores for promoting bone growth). The pores can also be positioned in any manner helpful for promoting bone growth, but in some embodiments, they are exposed at one or more surfaces of the implant 30 (e.g., any or all of the caudal surface, the cranial surface, the ventral surface, the dorsal surface, the left surface, or the right surface), which in some cases allows bone to access (e.g., grow into) the pores or allows for fluid to circulate within the implant.

In some embodiments, the plurality of pores is interconnected (e.g., each of the plurality of pores is fluidically interconnected with each of the other of the plurality of pores). In some embodiments, the implant 30 (or any particular column 32 of the implant) includes multiple porous structures 37 (e.g., multiple pluralities of pores), which in some cases are interconnected with each other, and in other cases are not interconnected with each other (although the pores within a plurality may still be internally interconnected). For example, in some embodiments, one or more columns 32 includes a porous structure, while one or more other columns do not include a porous structure (in some cases, expanding columns 32a include porous structures, but fixed columns 32b do not). By way of non-limiting illustration, FIG. 4B shows a fixed column 32b without a porous structure and expanding columns 34a that each include multiple porous structures 37 (a porous structure at the first outer region 36a, a porous structure at the second outer region 36b, and a porous structure at the inner region 38).

According to some embodiments, the pores of the porous structure 37 are defined by one or more strands 48. The strands can include any component or material capable of defining pores, such as one or more meshes, grids, filaments, foams, lattices, wires, grates, strings, springs, coils, webs, laces, filigrees, frames, frets, screens, reticulations, or other porous components or components that can define pores around them (whether they form strips, porous bodies, or other structures or configurations). By way of non-limiting illustration, FIG. 8A shows an implant 30 having multiple different types of strands 38 (which, in FIG. 8A, are generally interwoven, interconnected, filamentous, and spring-like). It may be helpful to note that, generally speaking, each cell in a bone must experience a specific amount of strain for that bone cell to be healthy. In this regard, the spring-like strands included in some embodiments of the implant allow for localized strain on a nano-scale rather than just global strain on a macro-scale (i.e., the stiffness per unit area would be the same for any region of the implant). It should also be noted, that some embodiments of the described implant use large voids to allow for a calculated global stiffness per global area that is different in concentrated areas of no voids.

Where the porous structure 37 includes one or more strands 48, the strands can have any configuration suitable for forming the porous structure. For example, in some embodiments, the strands (or portions thereof) are straight, curved, spiraled, twisted, zig-zagged, accordioned, branching, randomly dispersed, geometrically arranged, interwoven, interconnected, separate, independent, uniform, crossed with other strands, woven together, parallel to each other, diagonal to each other, placed with any suitable orientation with respect to each other, varied, or otherwise configured. In some embodiments, one or more of the strands are arranged in a spring-like configuration such that a stiffness is derived from compression of the strands. According to some embodiments, a combination of the material from which the porous structure is formed and the spring-like shape of the material results in a spring constant that yields the desired stiffness of the implant 30 (or the stiffness of one or more of the implant's columns 32 or load bearing regions).

In embodiments in which the porous structure 37 comprises strands 48, the strands can also extend through any portion of the porous structure and in any orientation. For example, the strands (individually or collectively) can extend through the whole porous structure or through only a portion of the porous structure.

Where a strand 48 includes a spring-like configuration, any shape can be used that results in spring-like action of the strand. For example, some embodiments of the strand include a configuration of a torsion spring, a coil spring, a conical spring, a compression spring, a helical spring, a leaf spring, a triangular spring, a rectangular spring, a diamond spring, a spring with another geometric configuration, a single-wire spring, a multi-wire spring, a spring grid, any spring as discussed in the PCT application publication WO2017100366A1 (entitled “Porous interbody spacer”) which is incorporated herein by reference in its entirety, or another type of spring. Furthermore, multiple strands operating as springs can operate dependently of each other (e.g., they are interconnected or coupled in a series configuration, as may be seen with multiple porous structures in a single column 32) or independently of each other (e.g., they are not interconnected, or they are coupled in a parallel configuration, as may be seen with porous structures in different columns). Even where strands are not interconnected (e.g., not physically joined together), strands can be interwoven with each other, such that one strand passes through a negative space not occupied by the other strand (but within the boundaries defined by such strand) without connecting to (or, in some cases, without even contacting) the other strand.

The porous structure 37 can have any number of strands 48, which can be part of one or more groups of strands having similar characteristics. For example, in some embodiments, the first strand is interconnected with additional strands (which, in some cases, have a similar size, shape, stiffness, design, or other configuration as the first strand). Similarly, in some embodiments, the second strand is interconnected with one or more additional strands (which, in some cases, have a similar size, shape, stiffness, design, or other configuration as the second strand). In some cases, maximizing surface area for bone attachment can benefit from having a substantial amount of exposed metal (or any other suitable material used to form the implant 30), which can (in some embodiments) be maximized by keeping the diameter or width of each strand at a minimum to increase the total number of strands. Indeed, some embodiments have between 1 and 10,000 strands, or any subrange thereof. In some cases, a substantial number of strands is generally preferred. For example, some embodiments have between 10 and 5,000 strands, 20 and 500 strands, 50 and 200 strands, or any other number of strands. Indeed, some embodiments (e.g., some lumbar embodiments) comprise about 200 strands ±30 strands, while some embodiments (e.g., some cervical embodiments) comprise about 80 strands ±30 strands.

In some embodiments, multiple different types of strands are used for different porous structures. For example, in some cases, thicker or stiffer strands are used for segments with a higher stiffness. By way of non-limiting illustration, FIG. 8A shows an implant 30 with a plurality of expanding columns 32a, each having at least one outer region 36 that includes a first strand 48 and an inner region 38 that includes a second strand 48, and the first strand is substantially thicker than the second strand so that the outer region has a substantially higher stiffness than the inner region.

In some embodiments, multiple porous structures 37 (e.g., of adjacent columns 32 or of a single column) are interconnected, but in some embodiments they are separated from one another. For example, some embodiments include one or more dividers 50 to separate porous structures from each other, to separate regions (e.g., an outer region 36 and an inner region 38) of a column 32 from each other, or to separate any other components or structures. Such dividers can have any suitable component for separating or delineating between components (e.g., one or more sheets, rods, barriers, gratings, meshes, lattices, nets, barriers, walls, or other dividing components). By way of non-limiting illustration, FIG. 8A shows an implant 30 with dividers 50 positioned between outer regions 36 and inner regions 38 of expanding columns 32a.

According to some embodiments (and as mentioned earlier), the implant 30 includes one or more anchor deployment mechanisms. In this regard, an anchor deployment mechanism can include any suitable mechanism that is configured to deploy one or more screws, pins, blades, or other anchors configured to fix the implant within the intervertebral space 22 (e.g., by pressing into or otherwise coupling to a bony endplate 24). As mentioned above, the anchor deployment mechanism of some embodiments is functionally coupled to an expansion deployment mechanism of the implant, such that deployment of one or more anchors also effectuates expansion of the expanding columns 32a. Although any suitable anchor deployment mechanism can be used, some embodiments use a mechanism as taught in U.S. Pat. No. 11,944,552 B2, entitled STAND-ALONE INTERBODY FUSION, filed Mar. 8, 2019, which is incorporated herein by reference in its entirety, or as taught in any of the provisional applications to which this disclosure claims priority. In some embodiments, such a mechanism is modified (in any suitable manner) to function without interfering with one or more columns 32, to interact with a coupler 40, or to otherwise operate in connection with the systems and methods described herein.

According to some embodiments, springs (e.g., for use in the expanding column 32a) are selected or formed to have particular stiffnesses or spring constants in accordance with one or more of the following equations:
Equation 1: In a compression spring, deflection is caused by twisting the wire diameter, and therefore the spring constant (k) is as follows:

$$k = \frac{G {d}^{4}}{8 {N}_{a} {D}^{3}}$$

Equation 2: Torsional stress of compression springs:

$${τ}_{∘} = \frac{8 DP}{π {d}^{3}}$$

The described systems and methods can be modified in any suitable manner. For example, although some embodiments of the implant 30 are specifically configured to be used in spinal surgery, some embodiments are configured to be used in other parts of the body (e.g., in joint replacements, bone grafts, or other types of surgery). Accordingly, some embodiments of the implant are not limited to being spinal implants. Indeed, in some embodiments, the implant is configured for use with one or more acetabular cups. Additionally, In some cases, the described systems and methods are modified for use with a femoral stem—allowing the expansion to occur in a radial direction.

In addition to the aforementioned features, the described systems and methods can include any other suitable feature. For example, some such additional features include one or more supplementary anchors (to anchor the implant 30 in place, such as by affixing the implant to one or more vertebral bodies 20). Other additional features can include protein coatings or other use of biological materials to stimulate bone growth.

Any and all of the components in the figures, embodiments, implementations, instances, cases, methods, applications, iterations, and other parts of this disclosure can be combined in any suitable manner. Additionally, any component can be removed, separated from other components, modified with or without modification of like components, or otherwise altered together or separately from anything else disclosed herein.

As used herein, the singular forms “a” “an”, “the” and other singular references include plural referents, and plural references include the singular, unless the context clearly dictates otherwise. For example, reference to a fastener includes reference to one or more fasteners, and reference to actuators includes reference to one or more actuators. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An orthopedic implant comprising:
a plurality of columns oriented to extend between a first bony surface and a second bony surface of a patient, the plurality of columns comprising:
a first support column; and
a first expandable column selectively couplable to the first support column, wherein a length of the first expandable column is selectively adjustable.

2. The orthopedic implant of claim 1, wherein a length of the first support column is fixed.

3. The orthopedic implant of claim 1, wherein a length of the first support column is selectively adjustable.

4. The orthopedic implant of claim 1, wherein the first support column comprises a first outer region, a second outer region, and an inner region, and wherein a stiffness of the inner region is different from at least one of a stiffness of the first outer region and a stiffness of the second outer region.

5. The orthopedic implant of claim 4, wherein the stiffness of the inner region is lower than the stiffness of the first outer region and the stiffness of the second outer region.

6. The orthopedic implant of claim 4, wherein the stiffness of the first outer region has a value that is within 10% of a value of a stiffness of the first support column.

7. The orthopedic implant of claim 4, wherein the stiffness of the inner region has a value that is at least 5% lower than a value of a stiffness of the first support column.

8. The orthopedic implant of claim 4, wherein the first outer region is configured to selectively couple to the first support column via a first coupler.

9. The orthopedic implant of claim 8, wherein the second outer region is configured to selectively couple to the first support column via a second coupler.

10. The orthopedic implant of claim 8, wherein the first coupler comprises at least one of a dovetail, a pin, and a band.

11. An interbody implant comprising:
a first surface configured to be disposed adjacent to a first bony endplate;
a second surface configured to be disposed adjacent to a second body endplate; and
a height extending between the first surface and the second surface,
wherein the interbody implant is configured to be selectively disposed in an insertion configuration and a deployed configuration, and wherein the height is greater in the deployed configuration than in the insertion configuration.

12. The interbody implant of claim 11, wherein the interbody implant comprises an expanding component configured to expand to increase the height of the interbody implant when the interbody implant is toggled from the insertion configuration to the deployed configuration.

13. The interbody implant of claim 12, wherein the expanding component of the interbody implant is compressed when the interbody implant is in the insertion configuration.

14. The interbody implant of claim 12, wherein the expanding component comprises an inflatable component configured to expand when selectively inflated by a user.

15. The interbody implant of claim 11, wherein the first surface is configured to conform to a topology of the first bony endplate.

16. A method of providing an interbody implant, the method comprising:
obtaining an interbody implant comprising:
a first surface configured to be disposed adjacent to a first bony endplate;
a second surface configured to be disposed adjacent to a second body endplate; and
a height extending between the first surface and the second surface,
wherein the interbody implant is configured to be selectively disposed in an insertion configuration and a deployed configuration, and wherein the height is greater in the deployed configuration than in the insertion configuration;
while the interbody implant is in the insertion configuration, inserting the interbody implant into an intervertebral space of a spine of a patient; and
when the interbody implant is disposed in the intervertebral space of the spine of the patient, toggling the interbody implant from the insertion configuration to the deployed configuration.

17. The method of claim 16, wherein the toggling the interbody implant from the insertion configuration to the deployed configuration comprises causing an expanding component of the interbody implant to expand to increase the height of the interbody implant.

18. The method of claim 17, wherein the expanding component is selectively couplable to a fixed component, and wherein the toggling the interbody implant from the insertion configuration to the deployed configuration comprises decoupling the expanding component from the fixed component.

19. The method of claim 18, further comprising recoupling the expanding component to the fixed component after toggling the interbody implant from the insertion configuration to the deployed configuration.

20. The method of claim 16, wherein the interbody implant further comprises:
a plurality of columns oriented to extend between a first bony endplate and a second bony endplate of the spine of the patient, the plurality of columns comprising:
a first support column; and
a first expandable column selectively couplable to the first support column, wherein a length of the first expandable column is selectively adjustable.
